(19) United States
(12) Patent Application Publication
(10) Pub. No.: US 2025/0280893 A1
(43) Pub. Date: Sep. 11, 2025

(54) AEROSOL PROVISION DEVICE

(71) Applicant: NICOVENTURES TRADING LIMITED, London (GB)

(72) Inventors: Darryl BAKER, London (GB); Nejat ERGUVEN, London (GB)

(21) Appl. No.: 18/858,900

(22) PCT Filed: Apr. 21, 2023
(86) PCT No.: PCT/GB2023/051046

§ 371 Date: Oct. 22, 2024

(30) Foreign Application Priority Data
Apr. 22, 2022 (GB) 2205854.9

(51) Int. Cl. A24F 40/53; A24F 40/65; A61M 15/06; G06F 21/32

(52) U.S. Cl. CPC A24F 40/53; A24F 40/65; G06F 21/32; A61M 15/06; A61M 2205/6009

(57) ABSTRACT

There is provided an aerosol provision device, for providing an aerosol for inhalation by a user, comprising: control circuitry for controlling an activation state of the aerosol provision device; and, age verification means for verifying an age of a user, wherein the control circuitry is arranged to update the activation state of the aerosol provision device from at least one inoperable mode to at least one operable mode in response to receiving a signal from the age verification means corresponding to an authorised user, and wherein the control circuitry is arranged to update the activation state of the aerosol provision device from at least one operable mode to at least one inoperable mode after a predetermined time period or predetermined use period of the device being in the at least one operable mode, wherein the predetermined time period or predetermined use period is variable.

### TECHNICAL FIELD

The present invention relates to an aerosol provision device, an aerosol provision system, a method of providing an aerosol to a user, and aerosol provision means.

### BACKGROUND

Aerosol-generating systems are known. Common systems use heaters which are activated by a user to create an aerosol by an aerosol provision device from an aerosol generating material which is then inhaled by the user. The device may be activated by a user at the push of a button or merely by the act of inhalation. Modern systems can use consumable elements containing the aerosol generating material. It can be desirable for the manufacturer to enable control over the activation of the systems. This may avoid the activation of the system in undesirable circumstances.

The present invention is directed toward solving some of the above problems.

### SUMMARY

Aspects of the invention are defined in the accompanying claims.

In accordance with some embodiments described herein, there is provided an aerosol provision device, for providing an aerosol for inhalation by a user, comprising: control circuitry for controlling an activation state of the aerosol provision device; and, verification means for verifying a user, wherein the control circuitry is arranged to update the activation state of the aerosol provision device from at least one inoperable mode to at least one operable mode in response to receiving a signal from the verification means corresponding to an authorised user, and wherein the control circuitry is arranged to update the activation state of the aerosol provision device from at least one operable mode to at least one inoperable mode after a predetermined time period or predetermined use period of the device being in the at least one operable mode, wherein the predetermined time period or predetermined use period is variable.

Such a device is able to verify a potential user of the device as being a suitable user to use the device. This may be as the user is recognised as being the owner of the device or based on the age of the user being suitable to use the device. The device may provide aerosols from age restricted aerosol generating material, it can be desirable therefore to provide a verification system that ensures the identity or age of a user is suitable for the device. This may be particularly of interest when the user is in a location or territory where being older than a specific age is a requirement for use of such a device. Recognition of the specific user may also operate as a proxy for authorisation of use of the device with specific aerosol generating material. The user may be identified or paired with the device at purchase or during set up or the like.

This allows the device to determine the identity or age of the user prior to a first usage. The device then requires a further authorisation after a predetermined time period or predetermined use period. In this way, an authorised user that age verifies themselves for use and then loses the device, does not enable the device to be used ad infinitum. Rather, this provides an increased in-use safety aspect for the device while avoiding unnecessary interference in the use of authorised users for a smoking session or other predetermined time period or the like. This therefore strikes a controllable balance between overly strict and overly lenient control over the usage of the aerosol provision device. The user can pre-program the device to require further authorisation checks on a regularity that suits the user. This protects the aerosol provision device from inappropriate use by inappropriate users.

The aerosol provision device of the present invention is able to operate in “offline” or “online” mode when performing verification of potential users. In this way, a valid user may operate the system in offline (unconnected to a mobile network or internet or the like) environment provided the user satisfies the criteria for operation. The user experience of the device is thereby improved. This can be achieved by including a memory in the aerosol provision device with which the verification means or control circuitry communicates.

In accordance with some embodiments described herein, there is provided an aerosol provision system, for providing an aerosol for inhalation by a user, comprising: an aerosol provision device comprising control circuitry for controlling an activation state of the aerosol provision device; and, verification means for verifying a user, wherein the control circuitry is arranged to update the activation state of the aerosol provision device from at least one inoperable mode to at least one operable mode in response to receiving a signal from the verification means corresponding to an authorised user, and wherein the control circuitry is arranged to update the activation state of the aerosol provision device from at least one operable mode to at least one inoperable mode after a predetermined time period or predetermined use period of the device being in the at least one operable mode, wherein the predetermined time period or predetermined use period is variable. vIn accordance with some embodiments described herein, there is provided a method of providing an aerosol to a user comprising: detecting, by control circuitry of an aerosol provision device, the conclusion of a predetermined time period or predetermined use period; updating, by the control circuitry, an activation state of the aerosol provision device from at least one operable mode to at least one inoperable mode; receiving, by the control circuitry, a signal from verification means associated with an authorised user; updating, by the control circuitry, an activation state of the aerosol provision device from at least one inoperable mode to at least one operable mode, wherein, the predetermined time period or predetermined use period is variable.

In accordance with some embodiments described herein, there is provided provision means, for providing an aerosol for inhalation by a user, comprising: control means for controlling an activation state of the aerosol provision means; and, verification means for verifying a user, wherein the control means is arranged to update the activation state of the aerosol provision means from at least one inoperable mode to at least one operable mode in response to receiving a signal from the verification means corresponding to an authorised user, and wherein the control means is arranged to update the activation state of the aerosol provision means from at least one operable mode to at least one inoperable mode after a predetermined time period or predetermined use period of the device being in the at least one operable mode, wherein the predetermined time period or predetermined use period is variable.

### DESCRIPTION OF DRAWINGS

The present teachings will now be described by way of example only with reference to the following figures:

FIG. 1 is a schematic view of an aerosol provision device according to an example;

FIG. 2 is a schematic view of an aerosol provision system according to an example; and,

FIG. 3 is a flow diagram according to an example.

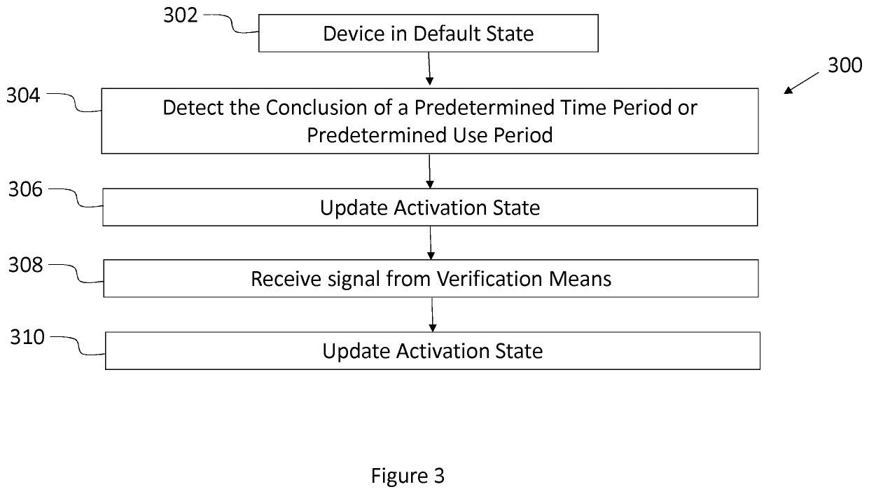

While the invention is susceptible to various modifications and alternative forms, specific embodiments are shown by way of example in the drawings and are herein described in detail. It should be understood, however, that the drawings and detailed description of the specific embodiments are not intended to limit the invention to the particular forms disclosed. On the contrary, the invention covers all modifications, equivalents and alternatives falling within the scope of the present invention as defined by the appended claims.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to aerosol provision systems, which may also be referred to as aerosol provision systems, such as e-cigarettes. Throughout the following description the term “e-cigarette” or “electronic cigarette” may sometimes be used, but it will be appreciated this term may be used interchangeably with aerosol provision system/device and electronic aerosol provision system/device. Furthermore, and as is common in the technical field, the terms “aerosol” and “vapour”, and related terms such as “vaporise”, “volatilise” and “aerosolise”, may generally be used interchangeably.

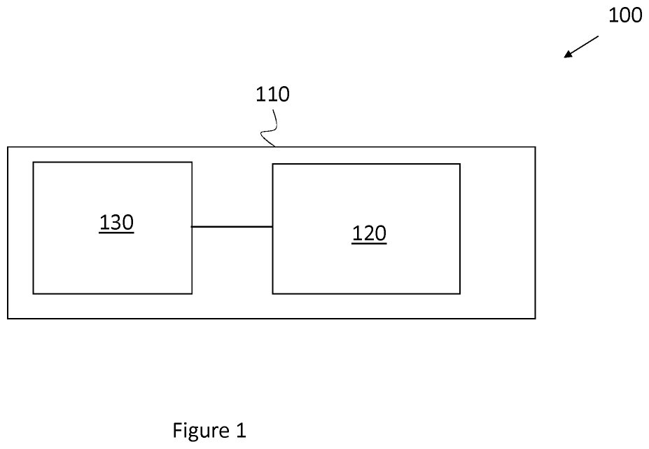

FIG. 1 illustrates a schematic view of an example of an aerosol provision device 100 according to the present invention. The aerosol provision device 100 comprises an aerosol provision device housing 110. The aerosol provision device 100 has control circuitry 120. The control circuitry 120 is arranged to control components of the aerosol provision device 100. The control circuitry 120 is arranged to control an activation state of the aerosol provision device 100. The aerosol provision device 100 comprises verification means 130 for verifying a user. The control circuitry 120 is arranged to update the activation state of the aerosol provision device 100 from at least one inoperable mode to at least one operable mode in response to receiving a signal from the verification means 130 corresponding to an authorised user. In this way, the device 100 may be operated when an appropriate user is detected.

The control circuitry 120 is further arranged to update the activation state of the aerosol provision device 100 from at least one operable mode to at least one inoperable mode after a predetermined time period or predetermined use period from receiving the signal from the verification means 120 corresponding to an authorised user and/or period of time of the device in at least one operable mode. In this way, the device 100 does not remain accessible ad infinitum, rather the user will be requested to re-satisfy age verification at a further point in time. This ensures an appropriate user is still using the device 100 at the second, or any further, checks.

The predetermined time period or predetermined use period is variable. In an example, the user can pre-program a timing (or use period) that is suitable for them. If they are in an environment wherein a second user obtaining the device is very slim (e.g. a person living alone), the time period may be greater than in an environment where that likelihood is higher (e.g. person with family). In this example, the user can still maintain safety over the device, but with as demanding a safety protocol as befits them. This provides a customisable balance for the user between safety and immediate accessibility.

Alternatively, the predetermined time period or predetermined use period may be related to the reliability of the user. In an example, with each successive satisfactory user verification, the time period or use period may be increased. In this way, reliable users (those that rarely do not have the device on their person) may be rewarded for having an inherently secure device. Less reliable users may have the time period reduced, to ensure that safety is preferentially satisfied over accessibility. This is very likely to be self-maintaining and reach a level of suitability that is bespoke to each user. This arrangement may also change over time, thereby allowing a user to increase their reliability and decrease the impact on use of the safety feature.

In an example, the predetermined time period or predetermined use period is at least one of: a predetermined number of puffs; at least one predetermined period of time in at least one particular operable mode; and, a predetermined number of use sessions. Each of these provides a useful and manageable counter by which to constrain use of the device. This may allow for e.g. a handful of smoking sessions to occur prior to verification. Again, the lower each of these, the less use an unauthorised user will gain from the device prior to the device returning to an inoperable state, and therefore the more inherently secure the device is against misuse.

In an example, the aerosol provision device is initially arranged in an operable mode. This allows some usage for a user prior to requesting verification and therefore prefers accessibility over security. In another example, the aerosol provision device is initially arranged in an inoperable mode. This allows no usage for a user prior to requesting verification and therefore prefers security over accessibility.

The verification means 130 may comprise at least one of: a face scanner; a projector scanner; a microphone; a fingerprint scanner; an iris scanner; and, a communication module for communicating with at least one of: a remote database; a remote computing arrangement; an onboard database; and, an onboard computing arrangement. Such a verification means 130 may be able to identify a user from e.g. a photo ID, or fingerprint scanner when compared against a database of users. Such a verification means 130 may be ale to age verify a user. In this way, an authorised, recognised user may be allowed to use the device 100 and an age-appropriate, recognised or unrecognised user may be allowed to use the device 100. In this way, a user of suitable age may not need to register with the device as a recognised user, rather the user is allowed to access the device as a result of being verifiably of a suitable age to use such devices.

In the example of a face scanner (or camera or the like), the verification means 130 for age, identity or the like may factor in aspects such as lines in the face, bags under the eyes, and prevalence of e.g. in males, facial hair to provide an age verification means. Such factors can be used to indicate a probable age range of the potential user of the device 100. The age verification means 130 may perform an analysis of the data taken of the user, and provide a signal to the control circuitry 120 indicating whether the user is of a likely suitable or likely unsuitable age for use of the device. Alternatively or additionally, the camera may be used to inspect a users identification, such as a passport to the like, to detect an age of the user. If there is an image on the identification, this can be compared against the user's face using the camera.

In an example, the verification means 130, which may be age verification means, receives signals from the control circuitry 120 to provide verification on the potential user and provides a notification to the user to perform verification. This verification may be age verification. The verification means 130 obtains a signal from the user and may process this in a number of ways. The verification means 130 may pass the signal to the control circuitry 120 for analysis or may analyse the signal directly.

In the example where the verification means 130 processes the signal directly, the verification means 130 may contain a database e.g. stored in a memory on board the device 100. The signal from the verification means 130 is compared to signals on the on board database. If the signal is associated with an authorised user, or a user of suitable age, the device 100 is updated into an operational state.

This on board database arrangement may be advantageous as the device 100 need not have a communications element in the device 100 to communicate with a remotely held database, and the device 100 need not be connected to a communications network to access a remotely held database prior to each use session. This may allow use of the device 100 in areas without connectivity. This may also provide a faster response than via communicating with a remote database.

In a different example, the database of authorised users or user age is held remotely, and the verification means 130 has a communication module to contact the database. The communication module may contact the database with a request for confirming user identity or age. The communication module, and therefore the age estimation module 130, is then provided with an age of the potential user, which is relayed to the control circuitry 120. User identity is used herein to refer to authorisation level of user. This may be either authorised or not authorised or may be specific levels of authorisation. The more authorised a user is deemed, the more functions that may be available for use with the device 100. Increased authorisation levels may be provided upon user registration of the device 100 which may include provision of ID or the like, user linking of the device 100 to a personal mobile device, user subscription to a service or the like.

This arrangement may be advantageous as the device 100 need not include a memory element for carrying the database and the database can be remotely updated ensuring the device 100 need not have the on board database regularly updated. In this way up-to-date user data can be provided to any relevant devices 100 as soon as the data is uploaded to the central database. In this way, all users can be provided with the updates without each needing to update their own device 100. The remote database may be a governmental database or the like for linking a property of a user to a known age (or identity) for that user, or for verifying the identification of a user where the identification contains an indication of age (or identity) for that user.

The verification means 130 may be able to communicate with an artificial intelligence computing arrangement to verify a user's age. This may be additional to, or alternatively to, the above options for verifying a user's age. The artificial intelligence computing arrangement may be arranged to process the signal received by the age verification means and provide an age estimation.

In the event that the user's signal is not recognised by the device 100, a communication element on the device 100 (for example, as part of the control circuitry 120 or age verification means 130) may communicate with a smart device and indicate that age verification is to occur. This may occur via a “Monzo”-style verification method. Such verification may occur using multiple authentication methods such as obtaining an image of a photographic identification document (e.g. passport or drivers licence or the like), and following this with obtaining a live video of the owner of said identification document. The video may involve the owner speaking a pre-requested phrase which may be unique to that individual, to further increase the difficulty of fraudulently satisfying the verification. The verification may utilise identity proofing, to assess claimed user identity against actual user identity. The photographic identification document contains an indication of age of the user. Multiple factor authentication is preferable to increase the reliability and safety of the system. Once this authentication is satisfied and the user is deemed of an appropriate identity, the user's identity may be stored suitably to enable future use of the device 100.

The control circuitry 120 may be arranged to update an activation state of the aerosol provision device 100 to an inoperable mode in response to receiving repeated signals from the verification means 130 associated with a non-authorised user. This provides a deterrent to potential abusers of the arrangement. A non-authorised user may be a non-recognised user or a user who is estimated to be of an inappropriate age for using the device 100.

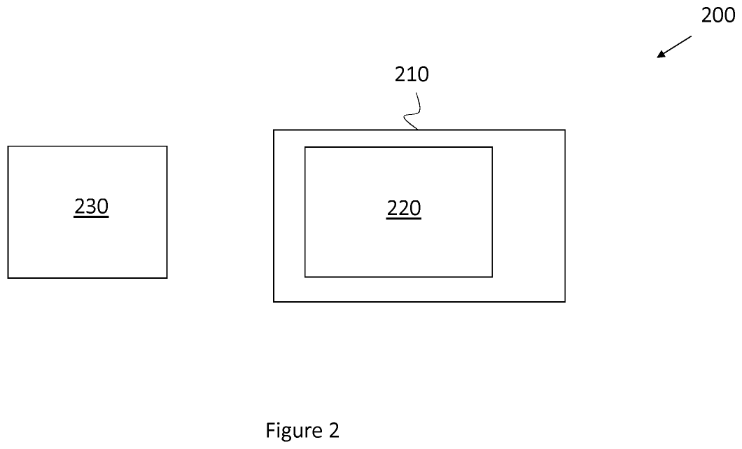

Referring now to FIG. 2, there is a shown a similar system 200 to the device 100 of FIG. 1. Similar features, to those features used in FIG. 1, are shown with the reference numerals increased by 100. For example, the device 100 of FIG. 1 is similar to the system 200 of FIG. 2. Similar or identical features may not be discussed for conciseness.

The system 200 of FIG. 2 has aerosol provision device housing 210 comprising the control circuitry 220. The system 200 also has verification means 230.

The verification means 230 may be separate to the aerosol provision device 210. The verification means 230 may be part of a mobile device or smart device belonging to the user of the aerosol provision device 210, with capability of receiving and processing signals from the user-such as via camera or microphone. This arrangement utilises the operability of a separate component thereby reducing the manufacturing requirements on the aerosol provision device 210. The smart device may be linked to the aerosol provision device 210 via an app or the like.

In an example, the aerosol provision device 210 can be more easily manufactured if elements such as the verification means 230 are not integral with the device 210. Furthermore, the verification means 230, which may be age verification means, being separate from the aerosol provision device 210 allows the system 200 to take advantage of other elements that can be accessed by a user. For example, the user's smart device may operate as a verification means 230. In the event that the user's signal is not recognised by the device 210, a communication element on the device 210 (for example, as part of the control circuitry 220) may communicate with the smart device and indicate that user, and/or user age, verification is to occur. This may occur via a “Monzo”-style verification method, as discussed above.

This arrangement allows suitable handling of a device that is used by multiple users, who may be of different age. The recognised users, or user of suitable age, are allowed to access suitable operational states for aerosol generating materials, while unrecognised user, or users of unsuitable age, are prevented from doing so. This ensures that suitability of use and accessibility are prioritised alongside user safety.

FIG. 4 shows a method 300 of providing an aerosol to a user. In the method 300, the device may start in a default state 302, which may be an operating state such that the device can be used without verification of a user.

The device then detects the conclusion of a predetermined time period or predetermined use period 304. This may be variable as explained above. The predetermined time period may begin when the device is changed from an operating mode to an inoperable mode. Control over this period can be used by the user to provide a bespoke level of safety for the device as explained above. This conclusion of the predetermined time period or predetermined use period is detected by the control circuitry. The device then updates the activation state of the device 306. This may occur via the control circuitry. The update may be from an operable mode to an inoperable mode.

The control circuitry then receives a signal from verification means associated with an authorised user 308. The activation state of the device is then updated by the control circuitry according to the signal from the verification means 310. This may be an update from an inoperable mode to an operable mode to enable the user to activate the device.

This method provides a user-friendly verification process that does not impede use for authorised users. In an example, it may provide a user-friendly age-verification process that does not impede use for authorised users. In particular, the method can provide a reactive system that benefits reliable users, and reduces the impact of the security aspects on reliable users, but that also protects users that require it. The method offers a balance between overly strict and overly lenient access protection for the device.

The method and device disclosed herein enable use of features of the device without requiring an arduous authorisation process—the process is only as arduous as is required considering the reliability of the user. This improves the user experience of the device and the safety of general use of the device.

The term “in response to” is used herein to indicate a second event (such as a signal or change of state of an aerosol provision device) that occurs subsequent to a first event. The second event may occur at a later time, after a predetermined time, or immediately after the first event.

The device and system herein are described as comprising several components that enable several advantages. The components may be disclosed as on-board the device or within the system. The components may be distributed and therefore not necessarily be located on-board the device. The functionality of the device can be provided by communicatively connected components, and such communication may be wireless, enabling such distribution. At which point it is reasonable to foresee that a distributed array of components will operate in the manner of the devices and systems disclosed herein. Components of the device or system may be contained in a further device such as a smartphone, computer, or remote server or the like.

In a particular example, the device disclosed herein may operate with a flavour pod which is replaceable in the device—this may be referred to as a consumable. The flavour may be any of tobacco and glycol and may include extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamon, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof.

When combined with an aerosol generating medium, the aerosol provision device as disclosed herein may be referred to as an aerosol provision system.

Thus there has been described an aerosol provision device, for providing an aerosol for inhalation by a user, comprising: control circuitry for controlling an activation state of the aerosol provision device; and, age verification means for verifying an age of a user, wherein the control circuitry is arranged to update the activation state of the aerosol provision device from at least one inoperable mode to at least one operable mode in response to receiving a signal from the age verification means corresponding to an authorised user, and wherein the control circuitry is arranged to update the activation state of the aerosol provision device from at least one operable mode to at least one inoperable mode after a predetermined time period or predetermined use period from receiving the signal from the age verification means corresponding to an authorised user, wherein the predetermined time period or predetermined use period is variable.

The aerosol provision system may be used in a tobacco industry product, for example a non-combustible aerosol provision system.

In one embodiment, the tobacco industry product comprises one or more components of a non-combustible aerosol provision system, such as a heater and an aerosolizable substrate.

In one embodiment, the aerosol provision system is an electronic cigarette also known as a vaping device.

In one embodiment the electronic cigarette comprises a heater, a power supply capable of supplying power to the heater, an aerosolizable substrate such as a liquid or gel, a housing and optionally a mouthpiece.

In one embodiment the aerosolizable substrate is contained in or on a substrate container. In one embodiment the substrate container is combined with or comprises the heater.

In one embodiment, the tobacco industry product is a heating product which releases one or more compounds by heating, but not burning, a substrate material. The substrate material is an aerosolizable material which may be for example tobacco or other non-tobacco products, which may or may not contain nicotine. In one embodiment, the heating device product is a tobacco heating product.

In one embodiment, the heating product is an electronic device.

In one embodiment, the tobacco heating product comprises a heater, a power supply capable of supplying power to the heater, an aerosolizable substrate such as a solid or gel material.

In one embodiment the heating product is a non-electronic article.

In one embodiment the heating product comprises an aerosolizable substrate such as a solid or gel material, and a heat source which is capable of supplying heat energy to the aerosolizable substrate without any electronic means, such as by burning a combustion material, such as charcoal.

In one embodiment the heating product also comprises a filter capable of filtering the aerosol generated by heating the aerosolizable substrate.

In some embodiments the aerosolizable substrate material may comprise an aerosol or aerosol generating agent or a humectant, such as glycerol, propylene glycol, triacetin or diethylene glycol.

In one embodiment, the tobacco industry product is a hybrid system to generate aerosol by heating, but not burning, a combination of substrate materials. The substrate materials may comprise for example solid, liquid or gel which may or may not contain nicotine. In one embodiment, the hybrid system comprises a liquid or gel substrate and a solid substrate. The solid substrate may be for example tobacco or other non-tobacco products, which may or may not contain nicotine. In one embodiment, the hybrid system comprises a liquid or gel substrate and tobacco.

In order to address various issues and advance the art, the entirety of this disclosure shows by way of illustration various embodiments in which the claimed invention(s) may be practiced and provide for a superior electronic aerosol provision system. The advantages and features of the disclosure are of a representative sample of embodiments only, and are not exhaustive and/or exclusive. They are presented only to assist in understanding and teach the claimed features. It is to be understood that advantages, embodiments, examples, functions, features, structures, and/or other aspects of the disclosure are not to be considered limitations on the disclosure as defined by the claims or limitations on equivalents to the claims, and that other embodiments may be utilised and modifications may be made without departing from the scope and/or spirit of the disclosure. Various embodiments may suitably comprise, consist of, or consist essentially of, various combinations of the disclosed elements, components, features, parts, steps, means, etc. In addition, the disclosure includes other inventions not presently claimed, but which may be claimed in future.

1. An aerosol provision device, for providing an aerosol for inhalation by a user, comprising:
control circuitry for controlling an activation state of the aerosol provision device; and,
verification means for verifying a user,
wherein the control circuitry is arranged to update the activation state of the aerosol provision device from at least one inoperable mode to at least one operable mode in response to receiving a signal from the verification means corresponding to an authorised user, and
wherein the control circuitry is arranged to update the activation state of the aerosol provision device from at least one operable mode to at least one inoperable mode after a predetermined time period or predetermined use period of the device being in the at least one operable mode,
wherein the predetermined time period or predetermined use period is variable.

2. An aerosol provision device according to claim 1, wherein the predetermined time period or predetermined use period is at least one of: a predetermined number of puffs; at least one predetermined period of time in at least one particular operable mode; and, a predetermined number of use sessions.

3. An aerosol provision device according to claim 1, wherein the aerosol provision device is initially arranged in an operable mode.

4. An aerosol provision device according to claim 1, wherein the aerosol provision device is initially arranged in an inoperable mode.

5. An aerosol provision device according to claim 1, wherein the verification means comprises at least one of: a face scanner; a projector scanner;
a microphone; a fingerprint scanner; an iris scanner; and, a communication module for communicating with at least one of: a remote database; a remote computing arrangement; an onboard database; and, an onboard computing arrangement.

6. An aerosol provision device according to claim 1, wherein the control circuitry is arranged to update an activation state of the aerosol provision device to a inoperable mode in response to receiving signals from the verification means associated with a non-authorised user.

7. An aerosol provision device according to claim 1, wherein the verification means is an age verification means.

8. An aerosol provision system, for providing an aerosol for inhalation by a user, comprising:
an aerosol provision device comprising control circuitry for controlling an activation state of the aerosol provision device; and,
verification means for verifying a user,
wherein the control circuitry is arranged to update the activation state of the aerosol provision device from at least one inoperable mode to at least one operable mode in response to receiving a signal from the verification means corresponding to an authorised user, and
wherein the control circuitry is arranged to update the activation state of the aerosol provision device from at least one operable mode to at least one inoperable mode after a predetermined time period or predetermined use period of the device being in the at least one operable mode,
wherein the predetermined time period or predetermined use period is variable.

9. An aerosol provision system according to claim 8, wherein the predetermined time period or predetermined use period is at least one of: a predetermined number of puffs; at least one predetermined period of time in at least one particular operable mode; and, a predetermined number of use sessions.

10. (canceled)

11. (canceled)

12. An aerosol provision system according to claim 8, wherein the verification means comprises at least one of: a face scanner; a projector scanner; a microphone; a fingerprint scanner; an iris scanner; a mobile device; and, a communication module for communicating with at least one of: a remote database; a remote computing arrangement; an onboard database; and, an onboard computing arrangement.

13. An aerosol provision system according to claim 8, wherein the control circuitry is arranged to update an activation state of the aerosol provision device to an inoperable mode in response to receiving signals from the verification means associated with a non-authorised user.

14. An aerosol provision system according to claim 8, wherein the aerosol provision device and the verification means are not integral.

15. An aerosol provision system according to claim 8, wherein the verification means is an age verification means.

16. A method of providing an aerosol to a user comprising:
detecting, by control circuitry of an aerosol provision device, the conclusion of a predetermined time period or predetermined use period;
updating, by the control circuitry, an activation state of the aerosol provision device from at least one operable mode to at least one inoperable mode;
receiving, by the control circuitry, a signal from verification means associated with an authorised user;
updating, by the control circuitry, an activation state of the aerosol provision device from at least one inoperable mode to at least one operable mode,
wherein, the predetermined time period or predetermined use period is variable.

17. A method according to claim 16, further comprising:
detecting, by the control circuitry of the aerosol provision device, the initiation of a second predetermined time period or predetermined use period;
updating, by the control circuitry, the second predetermined time period or predetermined use period to a predetermined time period or predetermined use period different from the predetermined time period or predetermined use period.

18. A method according to claim 17, wherein updating, by the control circuitry, the second predetermined time period or predetermined use period to a predetermined time period or predetermined use period different from the predetermined time period or predetermined use period comprises at least one of:
changing at least one predetermined period of time in at least one particular operable mode;
changing a number of puffs in the predetermined use period; and, changing a number of use sessions in the predetermined use period.

19. A method according to claim 16, wherein receiving, by the control circuitry, a signal from verification means associated with an authorised user comprises:
detecting, by verification means, a property associated with the user;
providing, by the verification means, a signal to the control circuitry.

20. A method according to claim 19, wherein detecting, by verification means, a property associated with the user, comprises at least one of:
scanning a face of the user; scanning a fingerprint of the user; scanning an iris of the user; detecting a voice of the user; and communicating with at least one of: a remote database; a remote computing arrangement; an onboard database; and, an onboard computing arrangement.

21. A method according to claim 16, wherein receiving, by the control circuitry, a signal from verification means associated with an authorised user comprises:
receiving, by the control circuitry, a signal from age verification means associated with an authorised user.

22. Aerosol provision means, for providing an aerosol for inhalation by a user, comprising:
control means for controlling an activation state of the aerosol provision means; and,
verification means for verifying a user,
wherein the control means is arranged to update the activation state of the aerosol provision means from at least one inoperable mode to at least one operable mode in response to receiving a signal from the verification means corresponding to an authorised user, and
wherein the control means is arranged to update the activation state of the aerosol provision means from at least one operable mode to at least one inoperable mode after a predetermined time period or predetermined use period of the device being in the at least one operable mode,
wherein the predetermined time period or predetermined use period is variable.
